(19) United States
(12) Patent Application Publication
(10) Pub. No.: US 2012/0315664 A1
(43) Pub. Date: Dec. 13, 2012

(54) ASSEMBLY AND METHOD FOR THE FILTRATION OF A LIQUID AND USE IN MICROSCOPY

(75) Inventors: Katja Friedrich, Nurnberg (DE); Walter Gumbrecht, Herzogenaurach (DE); Karsten Hiltawsky, Schwerte (DE); Peter Paulicka, Rottenbach (DE)

(73) Assignee: SIEMENS AKTIENGESELLSCHAFT, Munich (DE)

(21) Appl. No.: 13/575,651

(22) PCT Filed: Jan. 26, 2011
(86) PCT No.: PCT/EP2011/051064

§ 371 Date: Aug. 16, 2012

(30) Foreign Application Priority Data
Jan. 28, 2010 (DE) 10 2010 001 322.6

(51) Int. Cl. B01D 71/50; G01N 21/64; C12Q 1/04; B01D 63/00; B01D 61/00

(52) U.S. Cl. 435/34; 210/488; 210/650; 210/651

(57) ABSTRACT

An assembly and method are disclosed for the filtration of a liquid and the use thereof, wherein a supporting body is designed in a recess of a carrier and a filter membrane lies flat on the supporting body. The filter membrane and the supporting body are designed to be permeable to liquids and thus serve as filters, in particular for filtering tumor cells from blood. The carrier can having standard shapes of an object carrier for microscopy and the filtration residue on the filter membrane can be easily handled and examined in the microscope. As a result of the filter membrane lying level on the supporting body, the filtration residue can be particularly well examined microscopically.

### PRIORITY STATEMENT

This application is the national phase under 35 U.S.C. §371 of PCT International Application No. PCT/EP2011/051064 which has an International filing date of Jan. 26, 2011, which designated the United States of America, and which claims priority to German patent application number DE 10 2010 001 322.6 filed Jan. 28, 2010, the entire contents of each of which are hereby incorporated herein by reference.

### FIELD

At least one embodiment of the present invention generally relates to an assembly for the filtration of a liquid comprising a carrier, a filter membrane and a supporting body, wherein the supporting body is arranged and/or formed in a recess of the carrier. At least one embodiment of the present invention also generally relates to a method for the filtration of fluids with the assembly and to use for microscopic examination of residue remaining on the filter membrane.

### BACKGROUND

Microscopy is a widely used method in analysis. In particular in the field of “life sciences”, it is an indispensable tool in order, for example, to characterize tissue and cells. Object carriers have become the established “interface” between the medium to be examined and the imaging components of a microscope. These are glass plates measuring 26×76 mm (ISO 8255-2) with a thickness of from 1 to 1.5 mm. The objects are, for example, applied to the object carrier in a thin layer and can be covered with a cover glass, which, as a rule, measures 18×18 mm and is 0.16 mm thick. Objects are, for example, sections of tissue surrounded by a film of liquid.

Filtration is also a widely used technique, in particular for separating solids of different sizes from each other and/or from liquids. When microscopy and filtration are combined, following the filtration process, the filtration residue is examined microscopically. To this end, the filter medium, for example the filter membrane, has to be removed from the filtration device and placed on the object carrier. In particular with thin filter membranes, for example with filter membranes with a thickness of 10 μm and a diameter of 25 mm, this process requires considerable experimental skill and is very time consuming and susceptible to error, which, in practice, entails higher costs for a test procedure. In addition, manual interaction complicates standardization with respect to assuring a quality standard. Known problems with manual interaction are, for example, the partial destruction of the filter membrane and the accumulation of air bubbles between the filter membrane and the object carrier which hinders the subsequent microscopy.

To enable this process to be used routinely and inexpensively for medical diagnosis, for example during the examination of tumor cells filtered from a blood sample, it will be necessary to develop a simple and inexpensive solution, which can also be carried out by untrained personnel. Minimization of manual process steps also results in an improved potential for standardization and the avoidance of any impairment of the quality of the results.

### SUMMARY

At least one embodiment of the present invention discloses an assembly and/or a method for the filtration of liquids, which can be used with a high level of quality in standard methods and is easy to construct and handle and is inexpensive. In particular, an assembly and/or a method is disclosed for the filtration of liquids which is particularly suitable for subsequent microscopic examination. In this case, it must be possible to use the assembly in standard devices with standard holders, in order, for example, to carry out light microscopy or fluorescence microscopic examinations of filtration residue as standard, simply and inexpensively. In particular, it is an object to facilitate use for the recovery and examination of (circulating) tumor cells.

Advantageous embodiments of the assembly according to at least one embodiment of the invention and of the method for the filtration of a liquid and their use may be derived from the respective dependent subclaims. The features of the main claim can be combined with the features of the subclaims and the features of the subclaims can be combined with each other.

The assembly according to at least one embodiment of the invention for the filtration of a liquid or suspension comprises a carrier, a filter membrane and a supporting body. The supporting body is arranged and/or formed in a recess of the carrier. The filter membrane is arranged evenly and/or flat on the supporting body. In this context, flat means that the filter membrane is arranged in a planar area without peak-and-valley-like elevations.

### BRIEF DESCRIPTION OF THE DRAWINGS

Preferred embodiments of the invention with advantageous developments according to the features of the dependent claims are explained in more detail below with reference to the figures, but without being restricted thereto.

The figures show:

FIG. 1 a schematic representation of an assembly according to an embodiment of the invention in top view with a carrier, a supporting body and a filter membrane lying thereupon,

FIG. 2 a schematic sectional view through the assembly shown in FIG. 1,

FIG. 3 a detailed schematic view of the supporting body with channels and drainage holes, and

FIG. 4 a schematic sectional view through the supporting body shown in FIG. 3.

### DETAILED DESCRIPTION OF THE EXAMPLE EMBODIMENTS

The assembly according to at least one embodiment of the invention for the filtration of a liquid or suspension comprises a carrier, a filter membrane and a supporting body. The supporting body is arranged and/or formed in a recess of the carrier. The filter membrane is arranged evenly and/or flat on the supporting body. In this context, flat means that the filter membrane is arranged in a planar area without peak-and-valley-like elevations.

The structure of the assembly according to at least one embodiment of the invention enables a thin filter membrane to be applied to a carrier with a standard shape as a filter. During filtering, the supporting body provides mechanical support for the filter membrane, thus enabling large quantities of liquid to be filtered in a reasonable time. The level application of the filter membrane ensures, on the one hand, that there are numerous common support points between the filter membrane and the supporting body. This enables the filter membrane to be embodied as very thin without tearing when filtering large quantities of fluid with high rates of flow. Filter membranes, which can only be embodied as very thin, are, for example, filter membranes produced by particle bombardment from films with precisely defined through-pores or holes. Good support with the aid of the supporting body in the form of numerous, uniformly distributed support points is essential for the use of filter membranes of this kind as filters.

The carrier can be an object carrier, in particular for microscopy, which is made of glass or plastic, in particular polycarbonate. Both of these materials are inexpensive, easy to process and transparent in the visible range of light. The supporting body can be textured, in particular porous. The texture determines the number of support points for the filter membrane and enables filtered liquid to drain off after passing through the filter membrane. The supporting body can be made of plastic, in particular polycarbonate, or of a ceramic. Plastic is easy to texture and can, for example, be produced inexpensively and with a simple texture by way of injection molding. Slightly porous ceramics with a defined pore size are inexpensive to produce. The use of an object carrier as a carrier for the filter membrane facilitates simple handling and use in standard devices. Object carriers made of glass or plastic are very stable, both mechanically and chemically, which is important when preparing filtration residue prior to a microscopic examination, for example. A transparent carrier in particular enables use in light microscopy, in particular in transmission mode and direct light mode.

The carrier can have a thickness in the region of 1 to 1.5 mm, a length in the region of 75 to 76 mm and a width in the region of 25 to 26 mm. The filter membrane can have a thickness in the region of 1 to 20 μm, preferably in the region of 10 μm, and a diameter in the region of 25 mm. These dimensions make the carriers suitable for use in the most commonly used holdings in standard devices for object carriers. Good holding is ensured without slipping during an examination. Filter membranes with the specified dimensions are easy to produce, e.g. by particle bombardment, and are easy to arrange evenly on a carrier. In conjunction with the supporting body, they have sufficient stability to ensure that they do not tear or undergo any other kind of damage during efficient filter operation with high flow rates of fluids through the filter membrane.

The recess in the carrier can have the same size as the supporting body. This facilitates good holding of the supporting body in the carrier. On the other hand the supporting body can be produced integrally from the carrier material. In the second case, a permanently stable assembly is achieved. The supporting body can have a circular design and the filter membrane can also have a circular design. This facilitates use in systems with circular feed pipes and circular discharge pipes for fluids. A round embodiment also facilitates microscopy, because the entire circular region can be optically resolved in the microscope's field of view. This also supports use in a circular flow chamber, wherein this embodiment has advantages with respect to laminar flows and good cleaning possibilities.

As described above, the carrier and the supporting body can be embodied integrally as one piece. This makes production easier and increases stability during use. The edge region of the filter membrane can be secured to the carrier, wherein the filter membrane completely covers the recess in the carrier and in particular lies evenly on the supporting body in the region of the recess. The edge region of the filter membrane can be welded to the carrier. Complete covering of the filter membrane enables complete filtering of the liquid without any unfiltered liquid escaping through marginal regions of the recess. Good fastening, which can be liquid-tight, is provided if the filter membrane is welded to the carrier.

The supporting body can comprise channels formed on a side facing the filter membrane, which are in fluidic contact with the filter membrane. These channels facilitate good drainage of the filtered liquid from the filter membrane and hence good passage of liquid to be filtered through the filter membrane. The supporting body can in particular comprise channels which radiate from a central region of the supporting body in the direction of the carrier or in the direction of the edge region of the filter membrane, which in the following should also be understood to mean in the direction of the carrier. This enables the area covered by the filter membrane to be traversed by a large number of channels. Viewed from a mid-point of the supporting body, the number of channels in the direction of the carrier can increase, in particular with an increase in the number of channels with the square of the distance from the mid-point of the supporting body. This produces a high and uniform surface density of channels on the surface of the supporting body covered by the filter membrane.

In addition, the supporting body can comprise channels embodied on circular paths connecting the radiating channels fluidically to each other. An assembly with channels extending in a star shape and a circular shape facilitates good drainage of filtered liquid from the filter membrane. The network of circular channels extending in a star shape facilitates a high surface density of channels on the surface of the supporting body and the minimization of supporting body area without channels.

The channels can be embodied with a depth and/or width, or channel cross-sectional area which increases from a central region of the supporting body in the direction of the carrier. This has the same effect, or supports the effect, as that achieved by the previously described increase in the number of channels from the central region of the supporting body toward the edge region. In particular, the cross sections of the channels or their channel cross-sectional areas can increase with the square of the distance from the central region or from a mid-point of the supporting body or the supporting body surface (opposite the surface of the filter membrane). This means that the cross section or the area of the cross section is proportional to the square of the distance r from the mid-point of the supporting body or of the supporting body surface and hence in particular also of the filter membrane surface. Preferably, the increase in the channel cross-sectional areas can also be larger than the square of the distance r.

A further possibility for the embodiment of the channels is an embodiment with channel cross-sectional areas whose sum increases quadratically with the distance r of the channels from the mid-point of the supporting body and/or the membrane. It should be noted that, as a rule, the mid-point of the membrane surface or the membrane is identical to the mid-point of the supporting body surface or the supporting body. The increase in the channel cross sections with the distance from the mid-point of the supporting body also facilitates unimpeded drainage of filtered fluid in the direction of the edge region of the filter viewed from its center, wherein in the edge region more liquid flows through the filter than in the central region due to the larger area and the fluid coming from the central region.

In the region of the circumference of the supporting body, drainage holes for fluid which pass right through the thickness of the supporting body and/or of the carrier can be embodied in the supporting body and/or in the carrier. These can each be fluidically connected to one or more channels. Liquid collected and transported in the channels can be transported away from the filter membrane via the drainage holes from a front side to a rear side of the carrier. An unimpeded flow of liquid through and away from the filter membrane and hence good passage through the filter membrane and filtering at a high flow rate is facilitated. The assembly of the drainage holes in the edge region of the supporting body facilitates an embodiment of the drainage holes with a large or relatively large cross section without significantly restricting the stability of the filter membrane and the supportive action of the supporting body.

The filter membrane and the supporting body can comprise a planar common area with contact regions or direct mechanical contact points, hereinafter called the contact surface. Here, in particular, the filter membrane can have a maximum distance from the planar contact surface of less than 100 μm. The planar area facilitates the assembly of the filtration residue in an even plane, wherein, for example, during microscopy, good imaging is facilitated by means of good focusing on the filtered objects in the plane. In the region of the contact between the filter membrane and the supporting body, the supporting body can be embodied in the shape of bars, in particular bars with a triangular cross section (section along the height of the bars), with a width at the contact points with the filter membrane of less than or equal to 100 μm. Alternatively, the bars can also be embodied in the shape of columns (rectangular cross section along the height or longitudinal direction of the extension of the columns). The embodiment of the support points with a small width facilitates good drainage of the liquid through the filter membrane and a more uniform distribution of the residue on the filter. Minimization of the area with direct mechanical contact between the filter membrane and the supporting body can be achieved with a good supportive action of the supporting body, wherein an optimum between supportive action and minimal direct contact, i.e. good passage and drainage of the liquid through and from the filter membrane, is obtained.

The filter membrane can be a track etched filter membrane made of polycarbonate film and comprising holes with a diameter of micrometers, in particular 8 μm and a hole density of 1% to 80% (as the ratio of the perforated area to the overall area), in particular a hole density of 105 holes per square centimeter. Etched filter membranes can be produced with a precisely defined hole diameter without undue effort and have good mechanical stability with small thickness.]

The assembly can be thermally stable up to temperatures of 90° C. This facilitates the chemical and biochemical preparation of specific filtration residue prior to microscopic examination.

With a method for the filtration of liquids with the above-described assembly, blood can be used as the liquid, in particular blood mixed with a lysis buffer for the lysis of red blood cells, wherein cells are filtered out of the fluid. The cells filtered are cancer or tumor cells in the blood, in particular leukocytes as filtration residue, wherein no, or only a few healthy cells are retained by the filter membrane as filtration residue from the fluid. An embodiment of the filter membrane as a track etched filter membrane with an 8 μm hole size facilitates the separation of tumor cells from the fluid, without, or virtually without, the retention of healthy cells from the filtrate. The particularly uniform embodiment of channels or the channel network in the supporting body under the filter membrane facilitates good, uniform passage of filtered liquid, in particular uniformly through the entire filter membrane surface. This in turn facilitates a very uniform distribution of the filtration residue on the filter membrane surface, i.e. for example tumor cells, so that a subsequent examination and good optical resolution of, for example, individual cells is facilitated.

For better detection, the filtration residue can be stained after the conclusion of the filtration. In particular in the case of microscopic examinations, this simplifies the detection of, for example, tumor cells. Temperature stability up to 90° C. and good chemical resistance of the filter membrane, of the supporting body and/or of the carrier facilitate the preparation of the filtration residue for an examination, such as, for example the disruption of cells and the reproduction and marking of DNA or proteins. The use of lysis buffers facilitates the disintegration of cell walls and a PCR can be used for the reproduction of DNA, for example. Marking can take place, for example, via complementary DNA fragments with coupled dye, for example methylene blue. Alternatively, lysis is able to dissolve red blood cells and hence reduce the number of cells to be filtered. Leukocytes are not dissolved by lysis and tumor cells, which represent enlarged cells with a diameter up to 20 times that of normal leukocytes, can be separated from the healthy leukocytes (filtrate, which passes through the filter membrane) as filtration residue. Preparatory steps for the preparation for an optical examination of the tumor cells can encompass very complex chemical and thermal steps.

The assembly and/or the method can be used for the filtration and for microscopic examination of the filtration residue, in particular for light microscopy or fluorescence microscopic examination, wherein the extremely even filter membrane facilitates good and simple focusing on the filtration residue and a good, optically sharp depiction of the filtration residue. The small bar size of the supporting body and the resulting large area of channels in fluidic contact with the filter membrane hence available enables a uniform flow of a fluid through the filter membrane to be achieved and a uniformly distributed filtration residue to be recovered and examined, which is covered with very few or no unwanted particles. This is of particular advantage with microscopic examinations in transmission or direct light.

The advantages associated with the method for the filtration of fluids with the described assembly and its use are similar to the advantages described above with respect to the assembly.

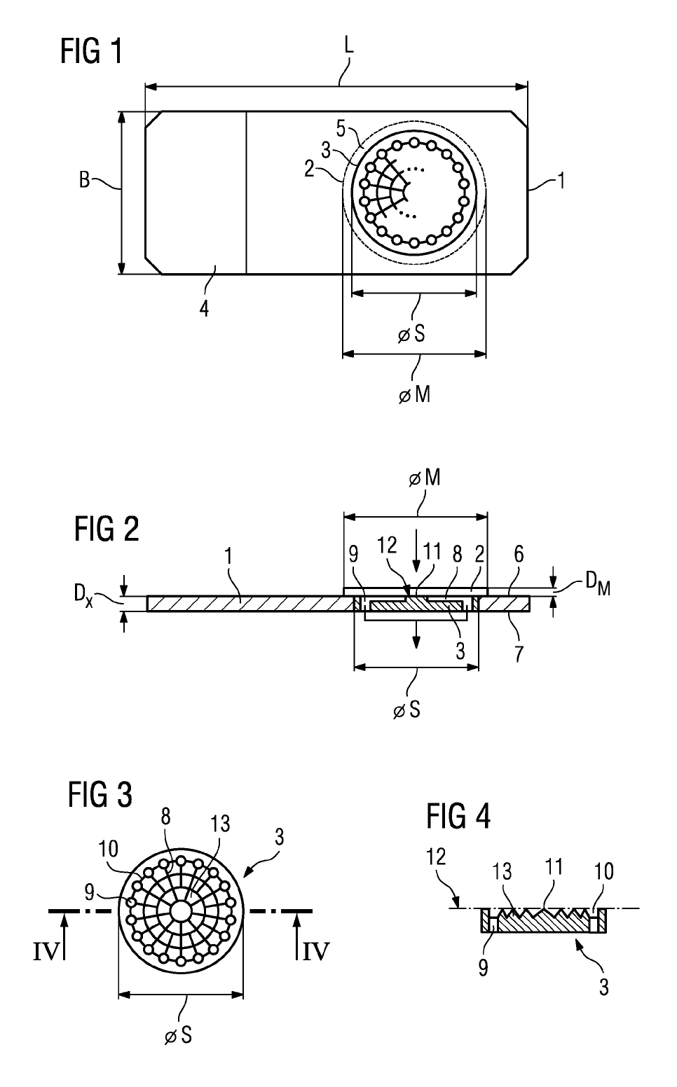

The assembly according to an embodiment of the invention shown in FIG. 1 comprises a carrier 1 and a supporting body 3 arranged in a recess of the carrier 1. The carrier 1 is embodied as even in the form of an object carrier for light microscopy. In a region disposed at a distance from supporting body 3, an area can be embodied as a grip 4 in that the surface is roughened, for example, in this region. Object carriers generally have a length L in the region of 76 mm and a width B in the region of 26 mm. Alternatively, object carriers can also have a length in the region of 75 mm and a width in the region of 25 mm. FIG. 2 shows a section along the length L of the carrier 1, wherein the carrier 1 has a thickness Dx. Generally, object carriers have a thickness Dx in the region of 1 to 1.5 mm. Standard object carriers with other sizes are also in use.

As FIGS. 1 and 2 show, a circular, film-type filter membrane 2 is arranged evenly on a front side 6 of the carrier 1 and the supporting body 3. The circular filter membrane 2 has, for example, a circular diameter ØM in the region of 25 mm and a thickness DM in the region of 10 μm. In the edge region 5, the filter membrane 2 is mechanically connected to the carrier, for example by welding or adhesion. The circular supporting body 3 is arranged below the filter membrane 2. The supporting body has, for example, a circular diameter ØS in the region of 23 mm and a thickness Dx corresponding to the thickness of the carrier. The filter membrane 2 lies evenly on the supporting body 3, wherein deviations from a planar contact surface between the supporting body 3 and filter membrane 2 can be, for example, maximum 100 μm. The supporting body 2 and the carrier 1 can be formed as one integral piece or the circular supporting body 3 can be arranged in a circular recess passing right through the thickness Dx of the carrier, in particular connected in a mechanically stable way to the carrier 2. In addition to circular shapes of the supporting body 3 and the recess, other shapes, for example rectangular or triangular shapes, are possible. A positive contact between the supporting body 3 and the recess of the carrier 1 is of advantage here.

As shown in FIGS. 1 and 2, channels 8, 10 are formed in the surface of the supporting body 3 on a front side 6. For purposes of simplicity, the channels 8, 10 are only indicated and not shown in full in FIG. 1. FIG. 3 is a detailed schematic view of a possible pattern of channels 8, 10 in the supporting body 3 although, for purposes of simplicity, with only a small number of channels 8, 10. The channels 8 extend in a star shape from the mid-point 11 of the circular shape of the filter membrane 2 or of the supporting body 3 in the direction of edge region 5, in the surface of the supporting body 3.

In order to keep the channel density of the channels 8 on the surface in the direction of edge region 5 substantially constant, the number of channels 8 increases in the direction of the edge 5 going from the mid-point 11. With an increase of the cross section of the channels 8 with the distance r from the mid-point 11, the maximum distance r is less than half the diameter ØS. The increase in the number and/or of the cross section of the channels 8 facilitates the uniform flow of a fluid to be filtered through the filter membrane 2. Alternatively or together with the increase in the number of channels 8 in the direction of edge 5, the channel cross sections or depressions in the carrier surface in the direction of the edge 5 can increase preferably quadratically with the distance r from the mid-point 11 or the sum of all cross-sectional areas of channels 8 lying on a circumference of a circle with a circle center 11 and radius r (distance r between the mid-point 11 and the circumference) can increase with the square of the distance r.

Drainage holes 9 passing completely through the thickness Dx of the carrier 1 or supporting body 3 are arranged close to the edge region 5 of the filter membrane 2 in the supporting body 3 or in the carrier 1 or in the contact region between the supporting body 3 and carrier 1. The channels 8 end in the drainage holes 9. Fluid flowing through the filter membrane 2 can come through the channels 8 and the drainage holes 9 from the front side of the carrier 6 and arrive at the rear side 7 of the carrier 1 and be transported away from there. Good uniform passage through filter membrane 2 and good filtering of the fluid are facilitated. In particular, a uniform pressure drop over the entire filter membrane surface is achieved.

The channels 8 are connected to each other by circular channels 10. The circular channels 10 result in an improved, in particular more uniform, fluid flow beneath the filter membrane 2. Similarly to the channels 8, the cross sections and/or number of the channels 10 can increase as the distance r from the mid-point 11 increases, in particular with the square of the distance r. Similarly to the channels 8, the sum of the cross sections of the channels 10 and/or of the channels 8 can increase as the distance r from the mid-point 11 increases, in particular with the square of the distance r.

In order to facilitate a uniform flow on the surface and good drainage of the fluid through the filter membrane 2 via the supporting body 3 in the direction of rear side 7 of the carrier 1, minimization of the direct contact between filter membrane 2 and supporting body 3 is advantageous. This can achieve uniform distribution of the filtrate on the filter membrane surface. Minimal mechanical contact on the surface between the filter membrane 2 and supporting body 3 is, for example, maintained if the channels 8, 10 are embodied in such a high number and density that, as shown in FIG. 4, only bars 11 are now embodied between the channels 8 and/or 10 on the surface of the supporting body 3. FIG. 4 shows a section through the supporting body 3 shown in FIG. 3 along a line of intersection IV-IV′, wherein, for reasons of better representation, only a small number of channels 10 and bars 13 is shown. A high number and density, and in particular a triangular shape form of the bars 13, as shown in FIG. 4, facilitate minimal direct mechanical contact between the filter membrane 2 and the supporting body 3 with high mechanical stability of the assembly. A particularly uniform fluid flow over the entire surface of the filter membrane 2, with the exception of the edge region 5, is achieved in this way.

The production of the assembly can be particularly simple and inexpensive if polycarbonates are used to produce the carrier 1 and the supporting body 3. In particular if the supporting body 3 and the carrier 1 are produced from one piece, channels 8, 10 can be milled into the surface of the supporting body. Alternatively, the channels 8, 10 can be formed by dead-mold casting or laser machining, for example. The drainage holes 9 can be produced, for example, by drilling, milling, laser machining or dead-mold casting. The filter membrane 2 can be produced from a film by particle bombardment, in particular as a track etched filter membrane from a polycarbonate film. In the edge region 5, the filter membrane 2 can be secured mechanically to the carrier 1 by adhesion or welding, for example.

Alternatively, if ceramic is used as the material for the supporting body 3, a porous layer can be formed on the surface of the supporting body 3, which, similarly to channels 8, 10, permits uniform drainage of a fluid. If the supporting body 3 is completely made of a porous material, the drainage holes 9 and channels 8, 10 can be provided by the porosity.

The use of track etched filter membranes formed from a polycarbonate film with a defined hole diameter facilitates the use of the assembly according to the invention for the filtration of, for example, tumor cells from blood. For example, with a hole diameter of, for example, 8 μm healthy cells in the blood (for example white and red blood cells) can substantially pass through the filter membrane 2, while tumor cells, which are too large and inelastic, are restrained by the filter membrane 2. The tumor cells are filtered out of the blood in this way and retained in the filter membrane (filtration residue). The uniform fluid flow over the surface of the filter membrane 2 facilitates filtering of the tumor cells in a form with which, after filtering, they lie substantially uniformly distributed on the filter membrane 2. This simplifies optical examination of the tumor cells. Alternatively to blood, it is also possible to filter other liquids and gases or solids contained in liquids.

The use of temperature-stable materials for the assembly according to the invention, at least in the region of up to 90° C., facilitates lysis cell disruption and the reproduction and marking of, for example, the DNA of the cells on the filter membrane 2. Alternatively, the cells themselves can, for example, be specifically stained with a dye. This simplifies an optical examination, in particular light microscopy or fluorescence microscopic examination of the filtration residue. The even, flat embodiment of the filter membrane 2 in an even, flat plane facilitates good focusing and depiction of the filtration residue.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. An assembly for the filtration of a liquid, comprising:
a carrier;
a filter membrane; and
a supporting body, the supporting body being at least one of arranged and formed in a recess of the carrier and
the filter membrane being substantially arranged at least one of evenly and flat on the supporting body.

2. The assembly of claim 1, wherein at least one of
the carrier is an object carrier for microscopy made of glass or of plastic; and
the supporting body is at least one of textured and porous and made of plastic or of a ceramic.

3. The assembly of claim 2, wherein the carrier has a thickness Dₓ in the region of 1 to 1.5 mm, a length L in the region of 75 to 76 mm and a width B in the region of 25 to 26 mm and wherein the filter membrane has a thickness D_{M} in the region of 1 to 20 μm, preferably in the region of 10 μm and a diameter ØM in the region of 25 mm.

4. The assembly of claim 1, wherein at least one of the recess in the carrier is at least one of the same size as the supporting body and embodied circular, and
the filter membrane is circular.

5. The assembly of claim 1, wherein at least one of the carrier and the supporting body are formed integrally as one piece, and
the edge region of the filter membrane is secured to the carrier, and wherein at least one of
the filter membrane completely covers the recess in the carrier and
the edge region of the filter membrane is welded to the carrier.

6. The assembly of claim 1, wherein the supporting body includes at least one of
channels on a side facing the filter membrane which are in fluidic contact with the filter membrane,
an increasing number of channels from a mid-point of the supporting body in the direction of the carrier and
channels embodied on circular paths connecting the channels fluidically with each other.

7. The assembly as claimed in claim 6, wherein the channels are embodied with at least one of at least one of a depth and width which increases from a central region of the supporting body in the direction of the carrier, with the square of the distance r or more increasing from the central region,
channels having channel cross-sectional areas, the sum of which increases quadratically with the distance r of the channels from the mid-point of the supporting body, and wherein that drainage openings for the filtrate which pass right through the thickness of at least one of the supporting body and the carrier are embodied in the region of the circumference of the supporting body in at least one of the supporting body and in the carrier, each of said drainage holes being fluidically connected to one or more channels.

8. The assembly of claim 1, wherein the filter membrane and the supporting body comprise a plurality of common contact points lying in a flat, even plane of the contact surface, wherein the filter membrane has a maximum distance from the planar contact surface of less than 100 μm and the supporting body in the region of the contact between the filter membrane and the supporting body is embodied in the shape of bars with a width in the region of 50 μm to 500 μm and/or in the shape of columns.

9. The assembly of claim 1, wherein the filter membrane is a track etched filter membrane made of polycarbonate film comprising holes with a diameter of micrometers and a hole density of 10⁵ holes per square centimeter.

10. The assembly of claim 1, wherein the assembly is thermally stable up to temperatures of 90° C.

11. A method for the filtration of fluids with an assembly comprising a carrier; a filter membrane; and a supporting body, the supporting body being at least one of arranged and formed in a recess of the carrier and the filter membrane being substantially arranged at least one of evenly and flat on the supporting body, wherein blood is used as a fluid, the method comprising:
filtering cells out of the fluid.

12. The method of claim 11, wherein the filtered cells filtered are cancer cells in the blood and no, or only a few healthy cells, are retained as residue from the fluid by the filter membrane.

13. The method as claimed in claim 12, wherein the filtration residue on the filter membrane is stained after the completion of the filtration.

14. A method comprising:
using the method of claim 11 for filtration and microscopic examination of the filtration residue, in particular light microscopy or fluorescence microscopic examination.

15. The method of claim claim 14, wherein the microscopic examination is performed by fluorescence microscopy.

16. The assembly as claimed 2, wherein the plastic is polycarbonate.

17. The assembly of claim 6, wherein the channels, on a side facing the filter membrane which are in fluidic contact with the filter membrane, are channels which extend from a central region of the supporting body in the direction of the carrier.

18. The method of claim 11, wherein blood mixed with lysis buffers is used as a fluid for the lysis of red blood cells.

19. The method of claim 14, wherein the method of claim 11 is used for light microscopy or fluorescence microscopic examination.
